(19) United States
(12) Patent Application Publication
(10) Pub. No.: US 2015/0150473 A1
(43) Pub. Date: Jun. 4, 2015

(54) Drug-Enhanced Neurofeedback

(71) Applicant: McLean Hospital Corporation, Belmont, MA (US)

(72) Inventors: Elizabeth Quattrocki Knight, Belmont, MA (US); Bruce Cohen, Lexington, MA (US)

(21) Appl. No.: 14/533,708

(22) Filed: Nov. 5, 2014

Related U.S. Application Data
(63) Continuation of application No. 13/127,296, filed on Jul. 13, 2011, now abandoned.
(60) Provisional application No. 61/111,147, filed on Nov. 4, 2008.

(51) Int. Cl. A61B 5/0482; G09B 23/28; G09B 5/02; A61K 45/06; A61B 6/03; A61K 31/42; A61K 31/198; A61K 31/197; A61B 5/055; G09B 5/04

(52) U.S. Cl. CPC A61B 5/0482; A61B 5/055; G09B 23/28; G09B 5/02; G09B 5/04; A61B 6/037; A61K 31/42; A61K 31/198; A61K 31/197; A61K 45/06

(57) ABSTRACT

The present invention relates to techniques and systems for training a subject to modify his or her own neuronal activity within a selected brain region.

### CROSS-REFERENCE TO RELATED APPLICATION

This application claims priority from U.S. Provisional Application Ser. No. 61/111,147, filed on Nov. 4, 2008, the content of which is incorporated herein by reference in its entirety.

### TECHNICAL FIELD

This disclosure relates to neurofeedback and, more particularly, to systems and methods that use specific agents to enhance neurofeedback techniques for modifying neuronal activity.

### BACKGROUND

Biofeedback, a technique developed in the 1960s, teaches individuals how to regulate autonomic bodily functions normally considered to be outside of the realm of conscious control. Biofeedback is founded on the concept that immediate and continuous feedback of information will amplify a conditioned response so that voluntary control can be achieved. Biofeedback can train people to consciously regulate autonomic functions such as heart rate, skin conductance, and bowel and bladder function and has even allowed individuals to modulate higher-level unconscious biological processes such as pain, athletic performance, and anxiety.

### SUMMARY

The techniques and systems described herein are based, at least in part, on the discovery that one can modify an individual's neuronal activity by administering agents that enhance learned changes in neural activation or neurotransmission to improve the efficacy of neurofeedback training. These agents can be, or include, substances such as, for example, partial agonists of the NMDA (N-methyl-D-aspartic acid) receptor. The administered agents improve NMDA receptor mediated neural activation or neurotransmission to enhance neurofeedback training of an individual.

In general, in one aspect, the invention features methods of training a subject to modify the subject's neuronal activity. The methods include administering to the subject an effective amount of an agent that enhances NMDA receptor-mediated neurotransmission; measuring levels of the subject's neuronal activity within a selected brain region over a predetermined period of time; and providing feedback to the subject in the form of a signal derived via imaging the selected brain region to enable the subject to modify the subject's neuronal activity within the selected brain region.

In these methods, the agent can be or include at least one of d-cycloserine, d-alanine, d-serine, glycine, or sarcosine. In certain embodiments, the first level of the subject's neuronal activity within the selected brain region can be measured using a brain imaging technology, e.g., at least one of functional magnetic resonance imaging, electroencephalography, magnetoencephalography, positron emission tomography, and single photon emission computed tomography. Data from the brain imaging can be analyzed in real time.

In some embodiments, the feedback is provided to the subject by presenting to the subject a representation of a level of the subject's neuronal activity within the selected brain region at a first time; providing the subject with a set of instructions configured to modify the subject's neuronal activity within the selected brain region; and presenting to the subject a representation of a level of the subject's neuronal activity within the selected brain region at a second time. For example, the representation can be or include a visual, olfactory, auditory, or tactile stimulus, and an interval between the first and second times can be about 100 ms to 10 seconds.

In these methods, the agent can be or include an indirect dopamine agonist, a cholinergic agent, a norepinephrine agent, a neuroendocrine agent a glial cell supporter, a glutamate/glutamine cycling facilitator, a nonspecific cognitive enhancer, a mitochondrial enhancing agent, and a histone deacetylase agent, alone or in combination with a NMDA receptor partial agonist. In various embodiments, the feedback can be provided in the form of a visual signal, a tactile signal, an olfactory signal, and/or an auditory signal.

In another embodiment, the invention features computer-readable media that store a computer program for training a subject to modify the subject's neuronal activity, the computer program including instructions for causing a computer system to: receive data indicative of levels of the subject's neuronal activity within a selected brain region over a predetermined length of time, wherein the subject is administered an effective amount of an agent that enhances NMDA receptor-mediated neurotransmission; and provide feedback to the subject in the form of a signal derived via imaging the selected brain region to modify the subject's neuronal activity within the selected brain region based at least on the received data. In certain embodiments, the computer program can further include instructions for causing a computer system to: present to the subject a representation of a level of the subject's' neuronal activity within the selected brain region at a first time; provide the subject with a set of instructions configured to help modify the subject's neuronal activity within the selected brain region; and present to the subject a representation of a level of the subject's neuronal activity within the selected brain region at a second time.

In another aspect, the invention further includes systems of training a subject to modify the subject's neuronal activity. These systems include at least a scanner configured to acquire data indicative of levels of the subject's neuronal activity within a selected brain region over a predetermined period of time; a processor configured to instruct a user to administer an effective amount of an agent that enhances NMDA receptor-mediated neurotransmission to the subject at the beginning of the predetermined period of time and to analyze the data acquired by the scanner; and an output device configured to provide the subject guidance to modify the subject's neuronal activity within the selected brain region.

In these systems, the scanner can be a magnetic resonance (MR) scanner, and these systems can be used to implement and carry out all of the methods and techniques described herein.

An interval between the first and second times during which the subject's neuronal activity within the selected brain region is presented can be about 0.1 to 10 seconds or more, e.g., 1 to 8 seconds, 3 to 6 seconds, or 4 to 5 seconds.

One or more agents can be administered to the subject, alone or in combination with an NMDA receptor partial agonist, to facilitate different aspects of real-time functional magnetic resonance imaging (rt-fMRI) neurofeedback. The agent can include a dopamine direct or indirect agonist, such as the stimulants ADDERALL®, CONCERTA®, STRATTERA®, or modafinil to enhance concentration and augment performance on the cognitive and/or mood induction tasks, and to indirectly modulate NMDA receptor mediated memory encoding and consolidation; a norepinephrine agonist, such as ephedrine, or phenylephrine to facilitate memory acquisition and encoding, and/or agents that reduce adrenergic neurotransmission such as propranolol, or other beta adrenergic antagonists, or clonidine, or other alpha-2-adrenergic agonists, to enhance relaxation techniques, reduce stress, and promote concentration during the rt-fMRI neurofeedback; a cholinergic agent, such as nicotine, milameline, sabcomeline, or xanomeline to facilitate memory encoding and consolidation and to indirectly augment NMDA receptor mediated plasticity; a nonspecific cognitive enhancer, such as dimebolin, caffeine, an agent that increases mitochondrial function, or a histone deacetylase agent to improve attention to tasks, to facilitate memory acquisition, and to promote the switch from short term memory to long term storage; neuroendocrine agents such as vasopressin and oxytocin analogues (Lixivaptan), cortisol, and thyroxine to indirectly enhance NMDA receptor neurotransmission, focus attention, and supplement memory formation; and glutamate/glutamine cycling facilitators such as N-acetylcysteine to support glial participation in synaptic plasticity and indirectly enhance NMDA receptor mediated neurotransmission either alone or in combination with an NMDA receptor partial agonist.

As used herein, “functional magnetic resonance imaging” refers to methods and techniques for monitoring the changes in a magnetic resonance (MR) signal over time that result from changes in the concentration of deoxyhemoglobin in a sample. The sample can be the brain, or one or more regions or circuits within the brain, of a living subject, such as a human, and changes in the MR signal can occur, for example, when the subject performs mental tasks.

As used herein, “real time” means within a time scale of a few seconds (e.g., about 1 second to about 10 seconds) or less (e.g., less than about 1 second).

The invention provides numerous benefits and advantages (some of which may be achieved only in some of its various aspects and implementations) including the following. In general, the invention allows a patient to experiment with and learn cognitive strategies that help ameliorate some symptoms associated with a disorder. The drug enhanced neurofeedback also allows the patient to assimilate the learned strategies on a long term basis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description, the drawings, and the claims.

### DESCRIPTION OF DRAWINGS

FIGS. 1A and 1B are schematics of a magnetic resonance system useful in the methods described herein.

FIG. 2 is a flowchart for performing a magnetic resonance experiment that uses neurofeedback.

FIGS. 3A-3C are representations of functional magnetic resonance images illustrated as statistical maps superimposed on anatomical magnetic resonance images of the human brain.

FIG. 3D is a graph that depicts an activation signal plotted as a function of time.

FIGS. 4A-4I are representations of functional magnetic resonance imaging signal activation during neurofeedback superimposed on anatomical magnetic resonance images of the human brain.

FIGS. 5A-5C are graphical representations of average signal activation as a function of time from a region of interest within a human brain.

FIGS. 6A-6C are a series of graphical representations of anatomical regions of interest, defined as the left and right insula, within a human brain.

FIGS. 7 and 8 are charts of activation in the insula of a human brain.

FIGS. 9A-9H are examples of functional magnetic resonance imaging signals from the insular region of a human brain during a neurofeedback training session displayed as a function of time.

FIG. 10 is a graphical representation of voxels that define a region of interest in the hippocampus of a human brain.

FIG. 11 is a chart of activation in the left and right hippocampi of a human brain.

FIGS. 12A-12D are graphs of magnetic resonance signals as a function of time from the voxels depicted in FIG. 10.

FIGS. 13A-13F are graphs of magnetic resonance signals as a function of time from voxels within the amygdala of a human brain.

FIG. 14 is a flow diagram depicting a study with a patient.

Like reference symbols in the various drawings indicate like elements.

### DETAILED DESCRIPTION

The invention is generally directed to new methods of enhancing neurofeedback combined with various brain imaging techniques. The feedback is typically in the form of a video display, sound, and/or mechanical output, e.g., a vibration, and enables the patient to quickly recognize and understand the level of neuronal activity in a brain region or regions, or in a brain “circuit” associated with a particular psychological or neuro-psychiatric disorder. In the new methods, one or more specific agents, e.g., partial agonists of the NMDA receptor, are provided to the patient just before or after a neurofeedback training session, to improve and enhance the effect and impact of the training session. This enhanced form of neurofeedback can be used to train an individual to attain certain levels, either reduced or elevated, of neuronal activity as therapy for various disorders such as substance abuse, anxiety, depression, epilepsy, obsessive compulsive disorders, learning disabilities, bipolar disorder, conduct disorders, anger and rage, cognitive impairment, migraines, headaches, chronic pain, autism, sleep dysregulation, posttraumatic stress disorder, and brain injuries.

### General Methodology

In general, the invention features new methods of training individual patients to modify their neuronal activity during neurofeedback sessions that utilize brain imaging. These new methods include administering an effective amount of an agent that enhances NMDA receptor-mediated neurotransmission, such as d-cycloserine (DCS), d-alanine, d-serine, glycine, or sarcosine, measuring levels of the patient's neuronal activity within one or more selected brain regions, or one or more brain circuits, over a predetermined period of time; and providing feedback to the patient to enable the patient to better modify his or her neuronal activity within the selected brain region, regions, or circuits. The selected brain targets being imaged are associated with a specific disorder. For example, for a patient with chronic pain, one would image the thalamus and the anterior cingulate. For mood disorders, one would image areas that process emotional stimuli such as the amygdala, insula, and/or regions in the prefrontal cortex. For obsessive compulsive disorder, one would target the ventral striatum, caudate nucleus, anterior cingulate, and orbitofrontal regions or circuits. To enhance learning and memory more generally, one would image the hippocampus, amygdale, and dorsolateral prefrontal cortex. Other examples of disorders and functions with their corresponding anatomical targets within the brain are listed herein.

Patients or subjects can be treated using these methods, for example, as follows. In some cases, the patient may need to visit a clinic or research facility a plurality of times, depending on the condition of the patient. During each visit, the patient is given an appropriate dose of an agent, such as DCS, and is then given one or more cognitive tasks and/or is presented with one or more sensory stimuli, while undergoing rt-fMRI scans. Real-time fMRI neurofeedback is provided to the patient based on images from a specific region or circuit of the brain. In some cases, initial rt-fMRI neurofeedback training runs are used to allow the patient to learn how to use the system, and then actual sessions are completed during which the patient is asked to complete specific cognitive tasks. In subsequent visits, the patient undergoes substantially the same procedures as described with respect to the first visit.

### Use of Imaging Modalities in Neurofeedback

Until recently, neurofeedback, a type of biofeedback, has utilized electroencephalogram (EEG) signal information to condition various response outcomes such as shifting the alpha, beta, and/or theta wave component of the EEG signal. Such neurofeedback has been applied to affect a behavioral outcome such as increased attention (Fox et al., Applied Psychophysiology and Biofeedback, 2005 30:365-373), enhanced musicality (Gruzelier et al., Prog Brain Res. 2006 159:421-431), and improved mood (Raymond et al., Brain Res. Cogn. Brain Res. 2005 23:287-292). This technique has also been applied to substance abuse disorders with varying degrees of success (Trudeau, Clin. Electroencephalogr., 2000 31(1):13-22). Because EEG measures averaged electrical activity at the scalp, traditional neurofeedback is neither anatomically specific nor able to monitor regions deep below the outer surface of the brain. Recent improvements in processing functional magnetic resonance imaging (fMRI) data and diminishing artifacts has now allowed real-time analysis of fMRI signals, with high spatial and temporal resolution, to replace EEG as the basis of the feedback signal and has opened up a whole new field of self-modulation of brain activation through real-time fMRI neurofeedback.

Functional MRI (fMRI) measures blood oxygen level dependent (BOLD) T2* weighted signal changes as an indirect way of visualizing neuronal activity in a localized brain area. Using real-time analysis of fMRI activation (rt-fMRI) changes as the neurofeedback signal, several studies have demonstrated that individuals can attain a degree of voluntary control over regional brain activation (see Weiskopf et al., Magn. Reson. Imag., 25(6):989-1003, 2007). In these studies, activation in a given brain region of interest can be analyzed in real time and presented back to the study subject in the form of either a visual or auditory stimulus. Each participant can be made aware of his or her brain activation as it is occurring with an approximate 3- to 6-second delay. The level of brain activation in the target region can be used to continuously modulate some easily discernable characteristic of a visual (e.g., still picture), video, audio, olfactory, tactile, or other stimulus being presented to the subject. With practice and the rt-fMRI feedback to guide them, subjects can intentionally increase and decrease regional brain activity. Sham feedback with signal information from areas other than the target region has not been shown to allow a similar voluntary control of the targeted brain areas.

Real-time fMRI neurofeedback teaches subjects to voluntarily regulate blood oxygen level dependent (BOLD) T2* weighted signal change by relaying to subjects in real time results of their self-modulation efforts. With practice, subjects can learn how to increase and decrease the BOLD signal in specific brain areas. Since BOLD activation is a measure of neuronal activity, rt-fMRI neurofeedback trains subjects to indirectly regulate neuronal activity in anatomically specified regions.

The regions that have been amenable to neuromodulation range from the cortical to the subcortical, including regions implicated in emotional state, cognition, mood, attention, anxiety, memory, reward pathways, and cravings. Real-time fMRI neurofeedback thus has wide applications in psychiatry as both a research and clinical tool and helps augment normal functioning as well. Characterizing the emotional and cognitive effects of increasing and decreasing activation in regional brain areas can help more accurately define which areas of the brain are the centers for cravings, attention, anxiety, mood, and impulse control. Additionally, because increasing or decreasing activation in one area of the brain will most likely indirectly change the activation in functionally connected areas of the brain, this technique can be used to identify, not just individual regions, but entire circuits of activity that contribute to psychopathology or addiction. Real-time fMRI directed neurofeedback can be individually customized to therapeutically ameliorate symptoms by targeting specific brain areas and by modifying circuits that represent suboptimal alternative pathways or by reinforcing beneficial pathways.

### Drug-Enhanced Learning

N-methyl-D-aspartate receptor (NMDA-R) mediated glutamate neurotransmission in the brain produces long term potentiation, a form of synaptic plasticity thought to be a key step in the cellular mechanism of learning and memory. Activation of the NMDA-R is multidimensional and depends on the synchronous activity of three components: (i) binding of the agonist, glutamate, (ii) occupancy at the glycine regulatory site by d-serine or glycine, and (iii) removal of the voltage dependent magnesium blockade through depolarization must all occur for the channel to open its pore and allow for the influx of calcium and other cations. The influx of calcium into the cell activates second messenger systems generating the downstream effects leading to long-term potentiation. The multidimensional requirements necessary for NMDA-R activation allow this receptor to play an integrative role in learning and memory.

Since NMDA receptors allow for the transmission of divalent cations, particularly calcium, into the cell, over stimulation of NMDA-R can lead to excitotoxicity and cell death. Thus, direct activation of the NMDA-R with agonists to the glutamate binding site can cause seizures and neuronal damage and should be avoided. However, indirect facilitation of NMDA receptor neurotransmission through the glycine regulatory site has the potential to enhance learning and memory without the risk of overstimulation and excitotoxicity. Agents that enhance NMDA receptor-mediated neurotransmission are referred to herein as partial agonists, and include d-cycloserine (DCS, originally used as an antibiotic to treat tuberculosis, subsequently discovered to have activity at NMDA receptors), d-alanine, d-serine, glycine, and a more recently identified agent called sarcosine. D-cycloserine, glycine, d-serine, and d-alanine all bind to the glycine regulatory site of the NMDA receptor, causing a conformational change in the NMDA receptor that allows for a greater influx of calcium ions. In contrast, sarcosine, a naturally occurring compound in the body, does not bind to the glycine regulatory site directly, but inhibits the type 1 glycine transporter, thereby increasing the concentration of glycine in the synapse and enhancing the functioning of the NMDA receptors. These partial agonist agents all augment the NMDA receptor functioning. In addition, a targeted dosing regime of these agents, such as DCS, administered just before, during, or just after a neurofeedback session, unlike chronic administration, can enhance learning of an associated task and have statistically significant effects on memory that can persist over time.

Administration and Dosage of Agents that Enhance NMDA Receptor-Mediated Neurotransmission

The agent DCS enhances NMDA receptor-mediated neurotransmission and is orally absorbed, with 70% to 90% bioavailability. Peak drug levels are achieved 4 to 8 hours after administration, it is metabolized primarily in the liver, the half-life of DCS is approximately 10 hours, and the pregnancy category for DCS is C. D-cycloserine is manufactured in 250 mg capsules under the trade name Seromycin® (Eli Lilly & Co., Indianapolis, Ind.). The dosage used for the present methods ranges from 100 mg to 500 mg or somewhat higher, e.g., 200 or 250 to 500 mg, and should be administered just before, during, or just after a brain imaging neurofeedback session. That regimen permits the drug to achieve peak potency in concert with learning that results during the 4 to 6 hours after the neurofeedback session.

Other agents that enhance NMDA receptor-mediated neurotransmission indirectly or that can augment the success of the rt-fMRI neurofeedback in other ways can be used alone or in combination with NMDA partial agonists to facilitate the functional utility of this technique as well. Other agents can include indirect or direct dopamine agonists, such as ADDERALL®, CONCERTA®, STRATTERA®, modafinil, or other stimulants to enhance the subject's concentration and augment performance on the cognitive and/or mood induction tasks, and to indirectly modulate NMDA receptor mediated memory encoding and consolidation; a norepinephrine agonist, such as ephedrine, or phenylephrine to facilitate memory acquisition and encoding and/or agents that reduce adrenergic neurotransmission such as propranolol, or other beta adrenergic antagonists, or clonidine, or other alpha 2 adrenergic agonists, to enhance relaxation techniques and promote concentration during the rt-fMRI neurofeedback; a cholinergic agent, such as nicotine, milameline, sabcomeline, or xanomeline to facilitate memory encoding and consolidation and to indirectly augment NMDA receptor mediated plasticity; a nonspecific cognitive enhancer, such as dimebolin, caffeine, a mitochondrial enhancing agent, or a histone deacetylase agent to improve attention to the tasks, to facilitate memory acquisition and to promote the switch from short term memory to long term storage; neuroendocrine agents such as vasopressin and oxytocin analogues (Lixivaptan), cortisol, and thyroxine to indirectly enhance NMDA receptor neurotransmission, focus attention, and supplement memory formation; and glutamate/glutamine cycling facilitators such as N-acetylcysteine to support glial participation in synaptic plasticity and indirectly enhance NMDA receptor mediated neurotransmission could be used to augment either real time fMRI neurofeedback training alone or in combination with NMDA receptor partial agonists.

Dosages of these other agents can be within their standard dosing ranges, as understood by one of ordinary skill in the art, and is typically higher than a chronic administration dosage.

Drug-Enhanced Real-Time fMRI

Single doses of an agent or agents that enhance attention, relaxation, and learning increase NMDA receptor-mediated neurotransmission in neurofeedback learning paradigms to enhance the success of the feedback if their administration is closely coupled to the training session.

The demonstration that rt-fMRI directed brain modulation can target anatomically specific regions and can elicit functional consequences suggests that this technique could have wide therapeutic applications both for treating pathological conditions and for improving normal brain functioning. Drug-enhanced rt-fMRI can thus both facilitate the training and enhance the persistence of the learning associated with the feedback process.

Direct and/or indirect NMDA receptor activation augments the success of rt-fMRI neurofeedback by facilitating the acquisition, improving the consolidation, and/or prolonging the persistence of the self-regulation achieved. Thus, the use of a cognitive enhancer with rt-fMRI neurofeedback can generate functional consequences that are therapeutic in many disease states and can improve functioning in normal conditions. This combined technique can be used to modulate many disorders, including, for example, the following disorders or functions and corresponding anatomical targets:
1. habituation (orbitofrontal, anterior cingulate, caudate, thalamic and striatal pathways) in obsessive compulsive disorder
2. memory and learning (hippocampus, amygdala, dorsolateral prefrontal cortex) in normal controls
3. extinction (amygdala, hippocampus, orbitofrontal cortex) in specific phobias
4. extinction (amygdala, anterior cingulate, posterior parietal lobes, medial prefrontal cortex, insula, and hippocampus) in post-traumatic stress disorder
5. relaxation (thalamus, prefrontal cortex, anterior cingulate, temporal lobes, basal forebrain/hypothalamus) in primary insomnia
6. suppression/activation of the default network in normal controls and in disease states such as schizophrenia, autism, and Alzheimer's disease
7. Mood (frontal cortical, insula, amygdala, subgenual anterior cingulate, caudate) in depression and bipolar disorder
8. attention (anterior cingulate cortex, prefrontal cortex, and striatum) in attention deficit hyperactivity disorder
9. Phonemic awareness and automaticity (parieto-temporal, occipito-temporal, dorsolateral cortex) in dyslexia
10. suppression of craving (insula and nucleus accumbens/striatal pathways) and improvement of impulse control (orbitofrontal cortex) in addictions
11. Positive (parieto-temporal cortex) and negative (dorsolateral prefrontal cortex) symptoms in schizophrenia
12. cognition (temporal lobes, hippocampus, frontal cortex) in Alzheimer's Disease
13. extinction of pain in chronic pain, phantom limb pain, and neuropathic pain (rostral anterior cingulate, thalamus)
14. anxiety reduction in anxiety disorders and in panic disorder (amygdala, insula, hippocampus, fronto-temporal regions)

### Magnetic Resonance Systems

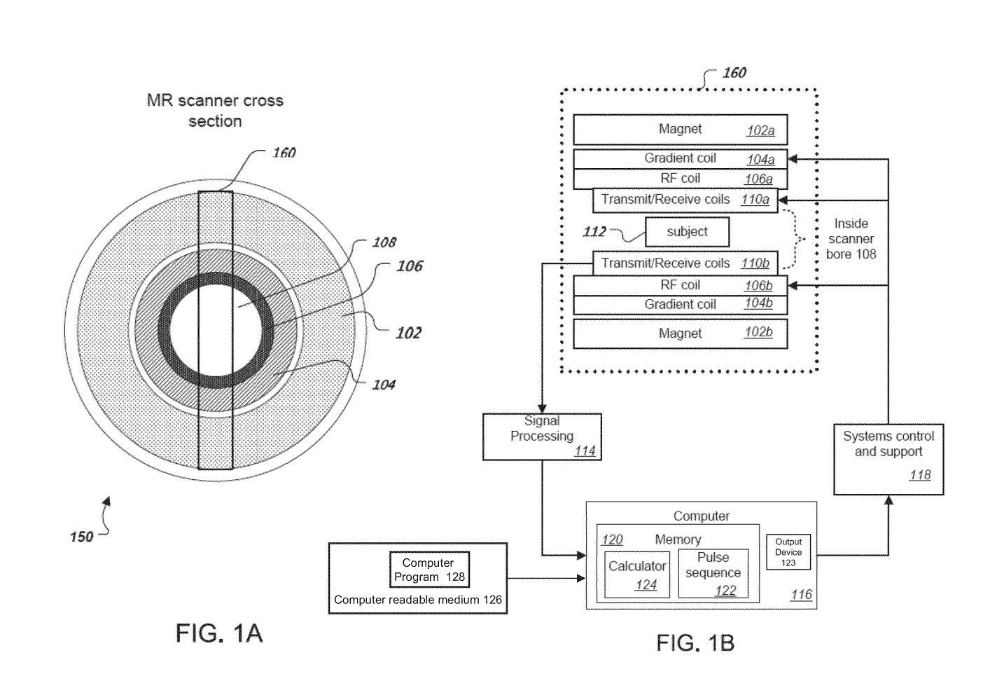

FIG. 1A shows a cross-section 150 of an exemplary magnetic resonance (MR) scanner that includes a magnet 102, a gradient coil 104, a radiofrequency (RF) coil 106 (e.g., a head coil), and a magnet bore 108. A slice 160 through the cross-section 150 is indicated and is described in more detail in conjunction with FIG. 1B.

The magnet 102 can have a field strength between about 0.5 Tesla and about 11 Tesla, for example, 1.5 T, 3 T, 4.7 T, 7 T, 9.4 T. The magnet 102 is designed to provide a constant, homogeneous magnetic field. The gradient coil 104 can include one, two, or three orthogonal, controller magnetic gradients used to acquire image or spectroscopic data of a desired slice by generating an encoded and slice-selective magnetic field. The RF coil 106 can be an integrated transceiver coil or can include both an RF transmit coil and an RF receive coil for transmitting and receiving RF pulses.

FIG. 1B is a block diagram of an MR system 100, which includes a slice 160 through the cross-section 150 of the MR scanner 150 as well as additional components of the MR system 100 and their connectivity. The slice 160 illustrates a top part of the magnet 102a and a bottom part of the magnet 102b, a top part of the gradient coil 104a and a bottom part of the gradient coil 104b, a top part of the RF coil 106a and a bottom part of the RF coil 106b. In addition, slice 160 shows a top part of transmit/receive coils 110a and a bottom part of transmit/receive coils 110b. These transmit/receive coils can surround a subject 112 (e.g., a human head) or can be placed above or below the subject or can be placed above and below the subject. The transmit/receive coils 110a and 110b and the subject 112 are placed within the magnet bore 108. The transmit/receive coils can have only a transmit functionality, only a receive functionality, or both transmit and receive functionalities. For example, a close-fitting smaller receive coil paired with a larger transmit coil can improve image quality if a small region is being imaged. Various types of coils (e.g., a head coil, a birdcage coil, a transverse electromagnetic or TEM coil, a set of array coils) can be placed around specific parts of a body (e.g., the head, knee, wrist) or can be implemented internally depending on the subject and imaging applications.

In FIG. 1B, the bottom part of transmit/receive coils 110b is connected to signal processor 114, which can include, e.g., pre-amplifiers, quadrature demodulators, analog-to-digital converters. Alternatively, the top part of transmit/receive coils 110a can be connected to signal processor 114. The signal processor 114 is connected to a computer 116. A systems controller 118, which is also connected to the computer 116, can include, e.g., digital-to-analog converters, gradient and RF power systems, power amplifiers, and eddy current compensation. The systems controller 118 is connected to the top part of the gradient coil 104a, the top part of transmit/receive coils 110a, and the bottom part of the RF coil 104b. Alternatively, the systems controller 118 can be connected to the bottom part of gradient coil 104b, the bottom part of transmit/receive coils 110b, or the top part of RF coil 104a.

The computer 116 controls the operation of other components of the MR system 100 (e.g., the gradient coil 106 and the RF coil 104) and receives MR data. In some embodiments, the computer 116 processes data associated with detected MR signals by executing operations, functions, and the like. The results of this data processing can be stored in memory 120, which can be random access memory (RAM), dynamic RAM (DRAM), static RAM (SRAM), etc. In some implementations, the memory 120 can include one or more storage devices (e.g., hard drives), individually or in combination with memory, for storing measurement data, processes, and other types of information.

In some embodiments, the computer 116 executes operations associated with a pulse sequence 122, and a calculator 124. The pulse sequence 122 is a set of instructions that are executed by various components of the MR system 100. The pulse sequence, which can reside in the memory 120, manages the timing of transmitted radiofrequency and gradient pulses as well as the collection of data.

The calculator 124, which can reside in the memory 120 and can be executable by the computer 116, performs operations (e.g., phasing, filtering, integrating, fitting, regressing) on the data collected from the MR system 100.

The system, e.g., the computer 116, includes an output device 123 that can provide a representation of neurofeedback data. The output device 123 can be a display device such as a liquid crystal display (LCD) or cathode ray tube (CRT) monitor that displays a visual rendition of the feedback signal, and is thus typically separate from the computer, and situated so that a patient within the MR system 100 can perceive the feedback signal. For example, the feedback signal can be represented by a change in the level of a line graph, or the color, shape, speed, or size of something that a patient can see on the display device. In some embodiments, the output device 123 can be an audio output device such as a speaker or a headphone. In such cases, the feedback signal can be represented by changing one or more of the tone, volume, or frequency of a sound that the patient can hear over the audio output device. In some embodiments, the output device 123 can be a tactile stimulator. In such cases, the feedback signal can be represented by varying the temperature, vibration, pressure, or some other characteristic of a tactile stimulus derived from the feedback data. In some embodiments, the output device 123 can be an olfactometer that adjusts an administered odor level based on the feedback signal. In some embodiments, the output device 123, in communication with the processor, instructs a user to administer an effective amount of an agent that enhances NMDA receptor-mediated neurotransmission to the subject.

In all embodiments, these output devices must be compatible with an MR system, and thus, are typically constructed without metal or other magnetic materials. The feedback signals are generated from the image data using known techniques and algorithms. See, e.g., deCharms, U.S. Published Patent Application Nos. US 2002/0103429 and US 2008/0001600, which are incorporated herein by reference in their entireties, and which also describe MRI systems and software that can be used to implement the methods described herein.

In some embodiments, the computer 116 communicates with a computer readable medium 126 that has encoded on it computer programs 128. The computer programs include instructions that can be executed by the computer 116. Even though FIG. 1B shows the computer readable medium to be external to the computer 116, the computer readable medium may be internal to the computer. For example, in some cases, the computer readable medium 126 with the computer program 128 may be a part of the memory 120 or other storage medium internal to the computer 116.

Techniques as disclosed in this specification can be implemented using the MR system 100, which can be any one of various commercial MR systems provided by, for example, Bruker BioSpin (Billerica, Mass.), Siemens Medical Solutions (Malvern, Pa.), or GE Healthcare Technologies (Milwaukee, Wis.), Philips Healthcare (Andover, Mass.).

### Magnetic Resonance Measurements

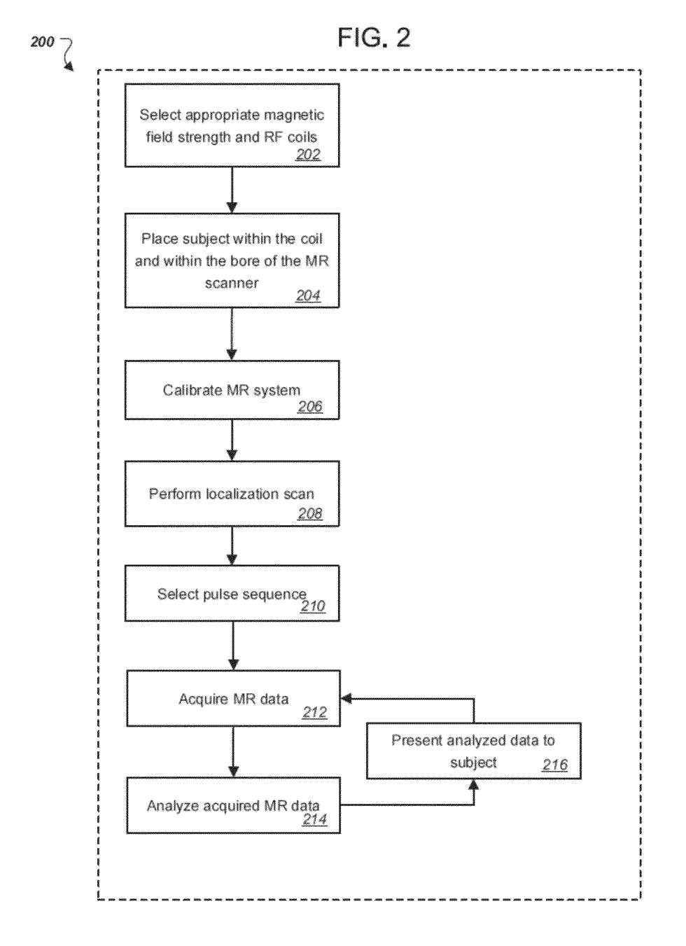

FIG. 2 is a flowchart that details the procedure in an example MR experiment.

A magnetic field strength and RF coils are selected that are appropriate for imaging the region of interest of the subject with the needed spatial and spectral resolution (step 202). Part of the subject 112 is placed within the RF coil and then placed in the bore 108 of the MR scanner (step 204). The MR system is calibrated (step 206), a process that can include, for example, checking the center frequency of RF transmission and shimming the magnetic field. A localization scan is then performed (step 208) to determine the anatomical placement of the subject within the coil and the MR scanner. The subject can be adjusted within the coil or MR scanner as needed. One or more pulse sequences are then selected (step 210) and MR data is acquired (step 212) for each sequence.

For a real-time fMRI experiment, the MR data is analyzed (step 214), which includes generating a BOLD signal time course. The analyzed data of the signal level in a selected brain region or circuit is presented to the subject (step 216) through either visual, auditory, tactile, or olfactory modalities in the MRI scanner to help them modulate the level of neuronal activation in the targeted region or circuit of the brain. A feedback loop is created by repeating steps 212, 214, and 216. Additional MR system adjustments can be made between data acquisitions.

### EXAMPLES

Results of rt-fMRI studies in the insula, sensorimotor cortex, amygdala, and hippocampal regions are provided in the following examples. All preliminary data were collected using a similar paradigm. After positioning and setup, the subject underwent a 30-second, 3-plane localizer scan for image prescription. A high resolution anatomic image set (T1 weighted MPRAGE-3D, resolution (RL, AP, SI) of 1.33×1×1 mm³ (TI=1100 ms, TR/TE=2100/2.74 ms, α=120°, FOV=170×256×256 mm³, 128×256×256 pixels, total imaging time 8 minutes and 59 seconds) was then acquired using a Siemens 3 Tesla MR scanner for coregistration of the functional data, which was acquired during a variety of subject tasks.

This image was immediately transferred to a Macintosh computer for reconstruction and Talairach alignment using Turbo-BrainVoyager™ (TBV) (Brain Innovations, Maastricht, the Netherlands). While this image was being processed, the fMRI slice stack was prescribed (27 oblique axial slices, 3.4 mm thick, no gap, parallel to the AC-PC line; the top slice was aligned with the top of the brain), and a second T1-weighted, high resolution distortion matched anatomical image set was acquired for later alignment of activation with neuroanatomy. Imaging parameters were as follows: a four-shot inversion recovery spin echo planar image (EPI) sequence, with a TI=2900 ms, TR/TE=7000/91 ms, 256×256 matrix, 220×220 mm² FOV, two averages, acquired in five concatenations for a total scan time of four minutes and 40 seconds.

TBV performed real-time processing of the acquired data. All functional data runs used the following parameters: TR/TE=1500/30 ms, 27 3.4 mm thick interleaved slices, parallel to the AC-PC line, no gap, an in-plane resolution of 64×64 with a 220×220 mm² FOV. The number of time points acquired varied depending on the task. The image acquisition used a modified EPI pulse sequence developed by Nikolaus Weiskopf (Weiskopf, et al., NeuroImage, 2003 19:577-56), which outputs an Analyze format file for each EPI volume within about 100 ms of acquisition to a special directory on the scanner's hard disk that is mounted via a server message block (SMB) on a remote Macintosh computer running TBV. As each new image was acquired, TBV performed motion-correction, slice-time-correction, and detrending on the image and used an incremental formulation of the general linear model to calculate both the instantaneous and cumulative activation levels in each voxel. Total latency between acquisition and presentation of feedback ranged between 100 ms and 6000 ms.

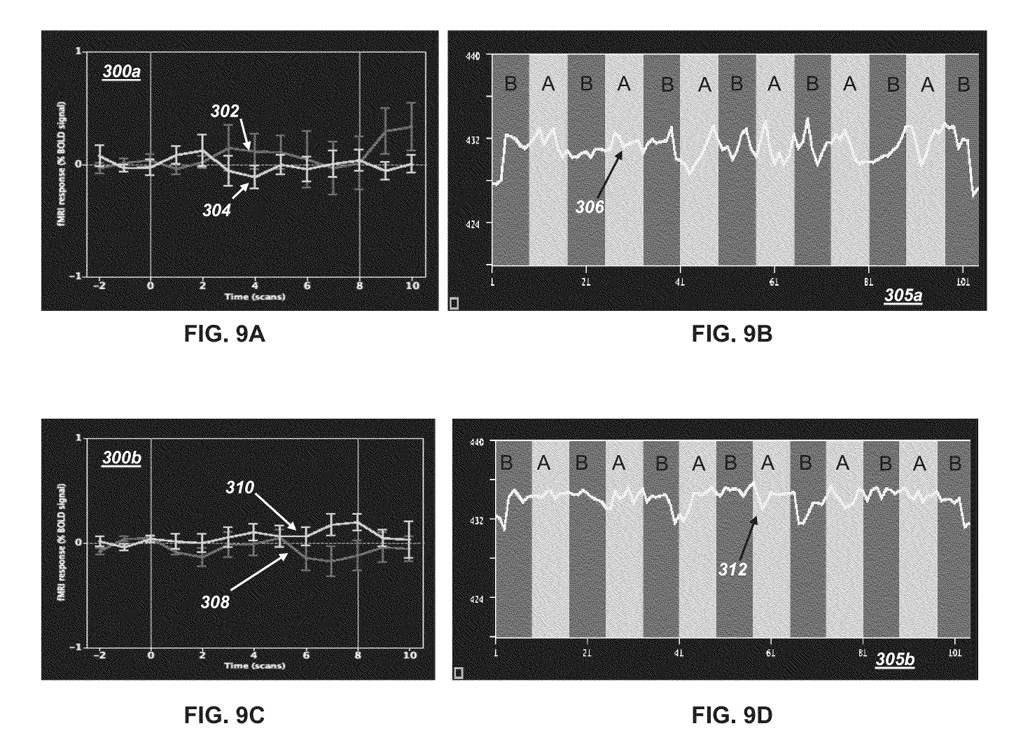

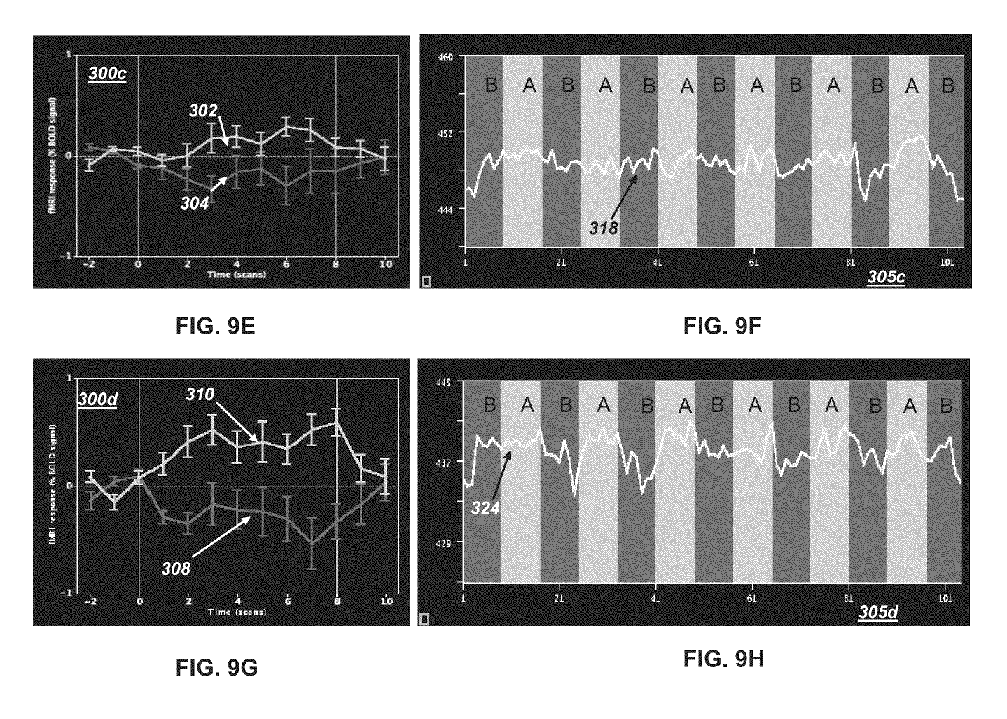

After using TBV to analyze and process in real time functional images that were acquired on a Siemens 3 Tesla (3 T) MR scanner, depictions of the level of neuronal activity were relayed back to the subject for operant training. The graphic display used to relay information back to the subject was a scrolling line chart superimposed on a background of alternating colored bars (for example, the line chart 305a shown in FIG. 9A). The display was projected onto a screen visible to the subject with a mirrored eyepiece affixed to the head coil. As the yellow line progressed across the background, the subject attempted to move the line up during the increase blocks and down during the decrease blocks.

### Example 1

### Real-Time fMRI Neurofeedback in the Sensorimotor Cortex

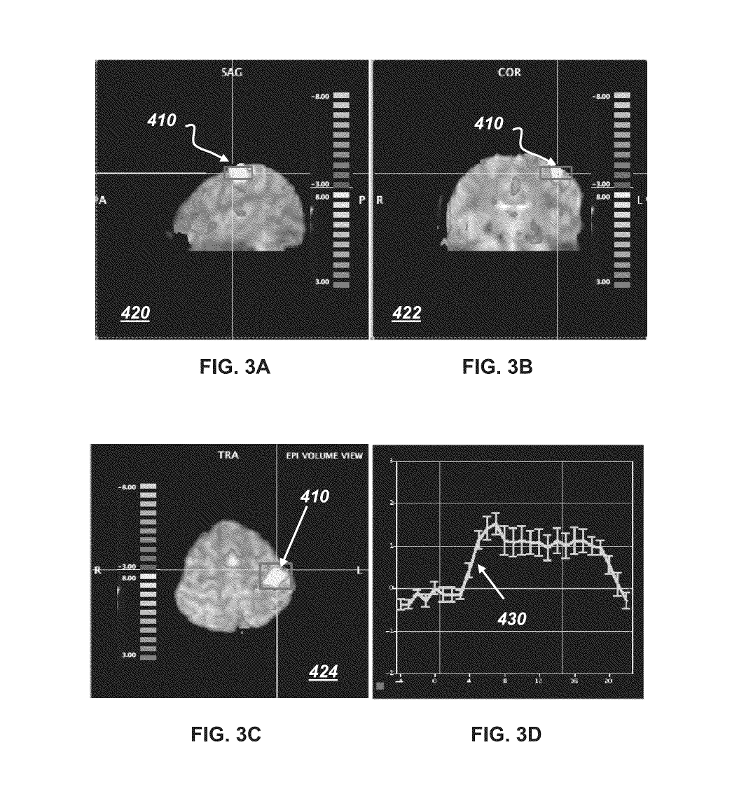

The successful neuromodulation of the sensorimotor cortex reported here, using the setup and protocol described below, validates these methods and provides a benchmark for evaluating graphic displays and other training protocols. A functional localizer scan was performed after anatomical scans were obtained, reconstructed and put into standard Talairach coordinates. FIG. 3A shows a sagittal image 420, FIG. 3B shows a coronal image 422, and FIG. 3C shows an axial image 424 obtained from a functional localizer scan that was performed after anatomical scans were obtained, reconstructed, and put into standard Talairach coordinates. Six cycles of right hand tapping, alternating with rest periods, were carried out by the subject while fMRI data were acquired. The echo planar images were acquired and processed using TBV while the subject was asked to tap the fingers of his right hand during the green ““ON” blocks and to remain still during the gray “OFF” blocks.

Since the actual finger tapping produces significant activation, when performed in conjunction with a localizer scan, this task can fine-tune the area targeted for neurofeedback. After a region of interest (ROI) was selected based on observed BOLD activation (e.g., by general linear model correlation analysis of the EPI images) during the functional localizer scan (FIG. 3), the signal from the ROI was relayed back to the subject via a scrolling line graph. A total of 13 blocks were each presented for 22.5 seconds. The activated voxels 410 were selected in the contralateral hemisphere and the activation signal from this region was then used to direct the real-time fMRI neurofeedback.

The subject was instructed that the object of the task was to increase the activation of the neurofeedback signal during the green periods and decrease the signal during the gray periods. The activation signal was presented as a yellow scrolling line graph traveling across a background of alternating green “ON” and gray “OFF” bars. The scrolling yellow line traveled at a rate such that each bar represented a time period of 22.5 seconds. The subject was asked to stay completely still, but was instructed to increase the signal during the “ON” periods by imagining the finger tapping task and to decrease the activation signal during the gray “OFF” by an alternative strategy such as an internal counting task. The subject participated in three rt-fMRI training runs. The duration of each run was 5-7 minutes. The signal from the selected region of interest was processed with TBV and relayed back to the subject via the projector screen within three seconds of acquisition (e.g., in a similar fashion as shown in line 306 of chart 305a in FIG. 9B). In FIGS. 3A-3C, statistical maps of fMRI signal activation are superimposed over an anatomical reconstruction of the subject's brain. FIG. 3D shows the average time course 430 of the active periods.

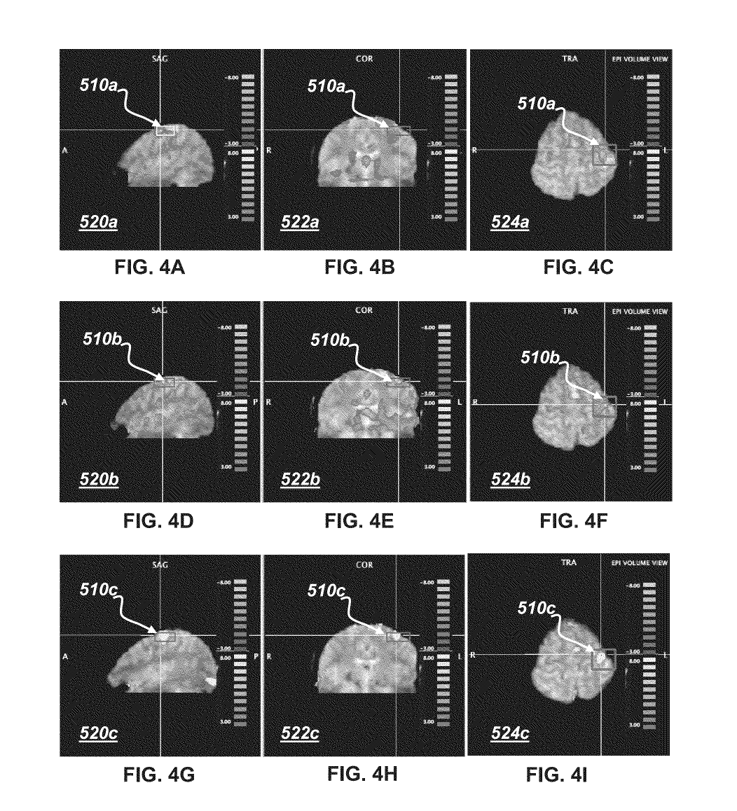

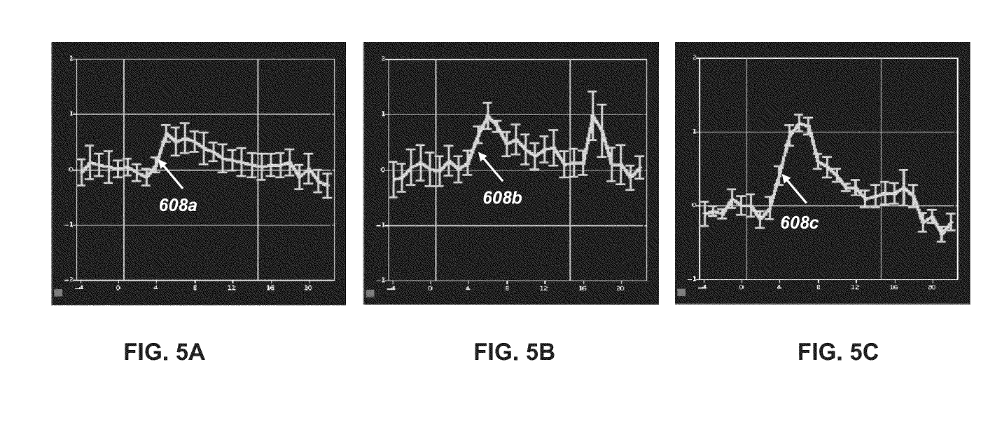

FIGS. 4A-4I display images and activation overlays while FIGS. 5A-5C show signal time courses for three sequential rt-fMRI neurofeedback training runs, respectively. A group of activated voxels 510a in the sensorimotor cortex is depicted in a box. FIG. 5A shows the event related average of the time course during the “increase” periods compared to baseline periods for the first run of FIGS. 4A-4C. Similarly, FIGS. 4D-4F and 5B are the activation and averaged signal, respectively, during the second rt-fMRI neurofeedback training session, and FIGS. 4G-4I and 5C are the activation and averaged signal, respectively, during the third rt-fMRI neurofeedback training session.

The activation is from the selected region of interest from the sensorimotor cortex that was activated during actual finger tapping. Statistical maps are superimposed on the functional images in FIGS. 4A-4I. Activation and averaged percent signal change during the first rt-fMRI training run that used only imagined finger tapping to control activation is shown in sagittal image 520a in FIG. 4A, coronal image 522a in FIG. 4B, and axial image 524a in FIG. 4C.

In FIGS. 5A-5C, the averaged signal from the green “ON” periods is presented as a time versus percent activation graph for multiple rt-fMRI neurofeedback training runs. The baseline is the average of the last portion of the “OFF” period. The increases in amplitude of the graph between runs 1 and 2 (i.e., the difference in the amplitudes of line 608a in FIG. 5A and line 608b in FIG. 5B) and between runs 2 and 3 (i.e., the difference in the amplitudes of line 608b in FIG. 5B and line 608c in FIG. 5C) indicate that the subject successfully increased his neural activation in the sensorimotor cortex by active neuromodulation.

By comparing, for example, the activation level in FIGS. 4A-4I (as evidenced by the grayscale of the activation overlay) or the height of the signal in FIGS. 5A-5C, it can be seen that the difference in activation between the green “ON” periods and the gray “OFF” periods increased with each rt-fMRI neurofeedback training run, demonstrating successful self-modulation of the activation signal in the sensorimotor cortex.

### Example 2

### Real-Time fMRI Neurofeedback in the Bilateral Insula

Other regions of the brain can also be used for neurofeedback training. In some cases, regions such as ones implicated in emotional states, learning, memory, anxiety, and cravings, including the insula, amygdala and bilateral hippocampal areas (described in Example 4), can be used.

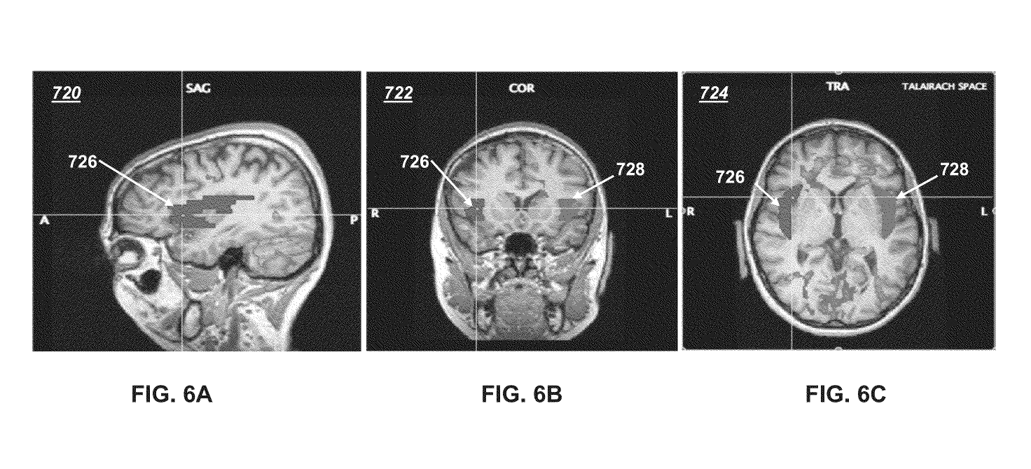

The insula lies within the cortex beneath the external operculum and is known to help integrate external sensory stimuli with internal emotional states and to assist in the processing of emotionally laden stimuli. In the study described below, instead of defining the region by an initial localizer scan, the anatomically defined regions of interest (region 726 for the right insula and region 728 for the left insula, as illustrated in sagittal image 720 in FIG. 6A, coronal image 722 in FIG. 6B, and axial image 724 in FIG. 6C) were used to delineate the region targeted during rt-fMRI neurofeedback.

Signals from both regions were relayed back to the subject during runs 1 and 2. During the third run, only the signal information from the right insula was used to direct self-modulation efforts. Learned enhancement of control over fMRI BOLD signal activation was observed, more for the right insula than the left. The difference between the averaged percent signal change between the increase blocks compared to the decrease blocks expanded, demonstrating modulation of signal activation in this area.

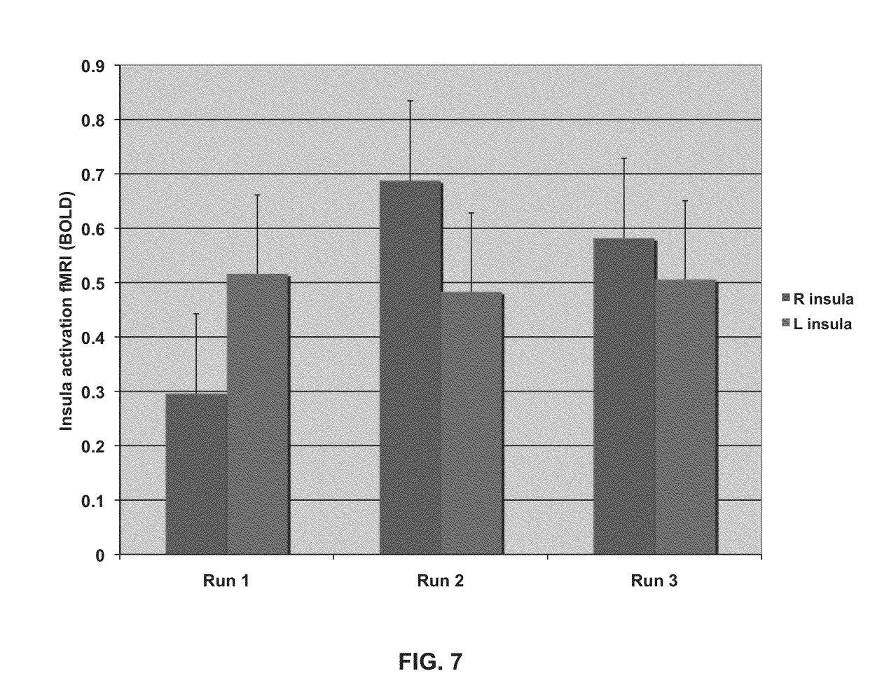

An initial high correlation of signal change was observed for the left insula during the first rt-fMRI neurofeedback training run, which may account for less observable changes between runs. As depicted in FIG. 7, however, no single voxel reached a statistically significant change during the first run, using q(false discovery rate (FDR))<0.05) as the threshold, yet repeat practice with additional rt-fMRI rt-fMRI neurofeedback training runs recruited more individual voxels to reach statistical significance.

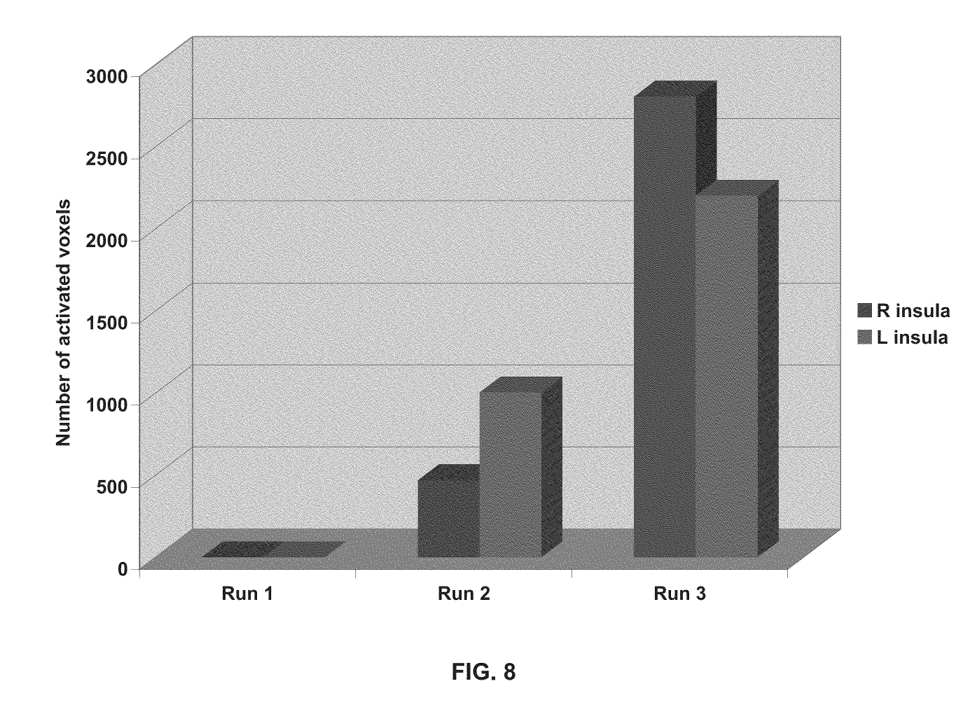

The right insula responded to rt-fMRI neurofeedback in all measurable respects. As illustrated in FIG. 8, the difference in percent signal change between the increase and decrease blocks grew larger, demonstrating improved self-control over BOLD signal activation. Additionally, the number of voxels within the region of interest that significantly correlated with the time course of the ON/OFF blocks increased with each subsequent rt-fMRI training run. The absolute number of statistically significant recruitment of voxels increased most dramatically between training runs 2 and 3, after the signal information from only the right insular region was transmitted back to the subject to direct self-modulation efforts.

### Example 3

### Real-Time fMRI Neurofeedback in the Anterior Insula

FIGS. 9A-9H depict the on-line image of the graphic display and the results from one rt-fMRI neurofeedback study targeting a region within the anterior insula. A subject was directed to increase brain activation in an anatomically-defined region of the insula. This brain activation was represented by a scrolling line graph (e.g., line 306 in chart 305a (FIG. 9B), line 312 in chart 305b (FIG. 9D), line 318 in chart 305c (FIG. 9F), line 324 in chart 305d (FIG. 9H)) during periods labeled “A” and decrease activation during periods labeled by “B” in charts 305a, 305b, 305c, and 305d. FIG. 9A depicts a graph 300a of the event-related average of the increase blocks (line 302) compared to the decrease blocks (line 304) and a graph 300b of the time course 306 of activation in the region of interest during the first run. FIGS. 9C-9D, FIGS. 9E-9F and FIGS. 9G-9H are the results from the second, third, and fourth rt-fMRI neurofeedback training runs, respectively. The subject in this study was able to improve self-control over activation with practice.

The activation can be viewed either on the native EPI images, or projected onto the 3D, Talairach registered anatomic image of the subject. ROIs were selected either geometrically, based on activated clusters in the current or previous acquisitions, or using anatomic definitions in Talairach space. The time courses from these ROIs can be output to a file for external processing, or directly presented in a number of formats from within TBV. For these experiments, all feedback was presented by graphing the time course, as acquired, (e.g., line 306 in chart 305a, line 312 in chart 305b, line 318 in chart 305c, line 324 in chart 305d) overlaid on a colored background indicating the epoch (increase activation or decrease activation) as shown in FIGS. 9B, 9D, 9F and 9H as “A” and “B” blocks.

The results from this preliminary rt-fMRI neurofeedback study demonstrate that the hardware and software neurofeedback setup can facilitate enhancement of self-control over regional brain activity. Based on these results, our current system appears to facilitate the self-modulation of the insula, which is a deeper cortical structure known to be involved in emotional states and learning and memory.

### Example 4

### Real-Time fMRI Neurofeedback in the Bilateral Hippocampus

The hippocampus is the primary locus for memory formation and is an important neural substrate for learning. Connections between the hippocampus, the amygdala, and other areas of the cortex make the hippocampus an important neural substrate contributing to learning, memory, cravings (Wang, Z., et al., J Neurosci, 2007 27(51):14035-14040), mood, fear and anxiety. In subjects with alcohol dependence, the amygdala/hippocampal area was the primary region activating in response to cue-induced craving (Schneider, F., et al., Am J Psychiatry, 2001 158(7):1075-1083). The important role the hippocampus plays in the circuitry underlying both cognition and emotional states makes this area useful for self-modulation using rt-fMRI.

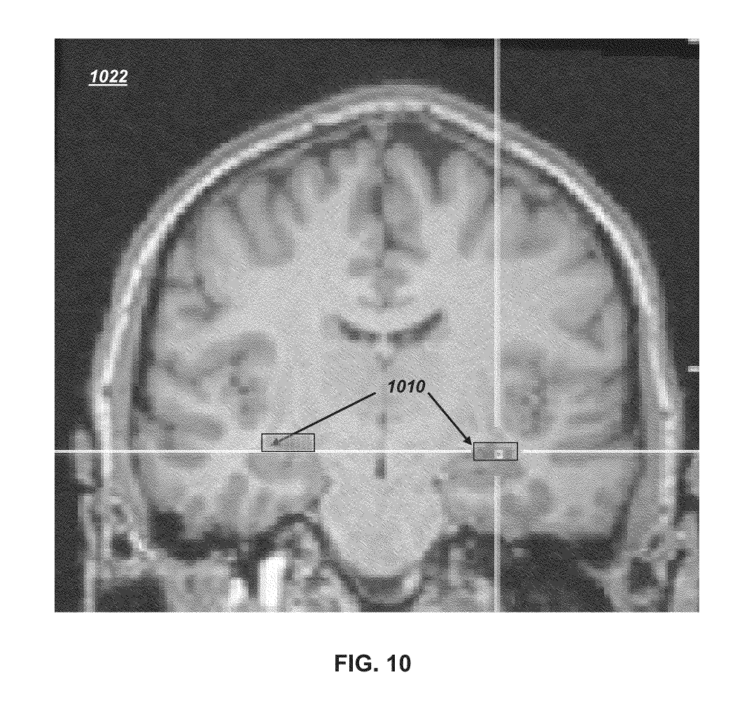

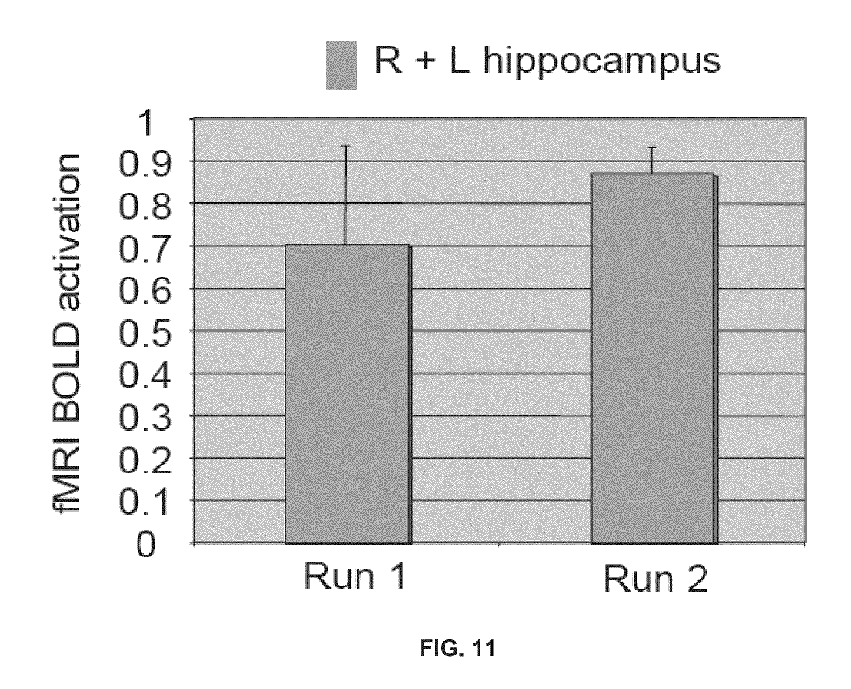

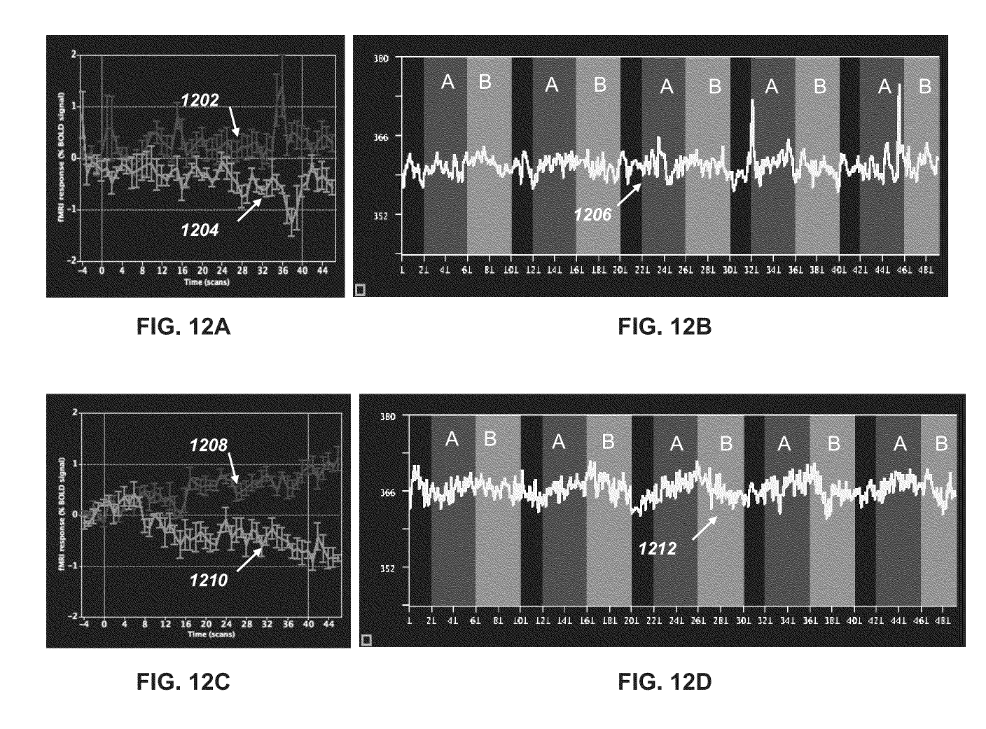

Similar to the approach used with the sensorimotor study described above, a region of interest was selected based on a functional localizer scan for neurofeedback of the hippocampus. Instead of finger tapping during activation periods however, the subject mentally reviewed memories of favorite songs. The region selected is depicted by the light blue area 1010 in FIG. 10. After two training runs, enhancement of control over regional activation in the hippocampus was observed. The difference between the average percent change in signal was increased between activation blocks compared to deactivation blocks and the number of voxels reaching statistically significance within the defined ROI increased during the second training run compared to the first run. The data from rt-fMRI of the hippocampus is shown in FIGS. 11 and 12A-12D.

FIG. 11 illustrates self-modulation of the hippocampus by a single subject after two training sessions of rt-fMRI. The difference in the percent signal change between the increase and decrease periods expanded during two training runs. Although the difference in the percent signal change between the first and second training run is not significant, 28.6% of the voxels within the targeted region of interest activated significantly (using a threshold of q(FDR)<0.05) in the second run, compared to the first run when none of the voxels within the region reached statistical significance.

FIGS. 12A-D demonstrate enhancement of brain activation control in an anatomically-defined bilateral hippocampal region within a single subject who is using rt-fMRI neurofeedback. Signal 1202 in FIG. 12A corresponds to the event-related average graphs of the measured fMRI signal during the increase periods (labeled “A” in FIG. 12B) and signal 1204 corresponds to measured fMRI signal during the decrease periods (labeled “B” in FIG. 12B) during the first run. FIG. 12B depicts the time course of the actual signal during the first run, shown as a yellow line graph. The subject was instructed to try to increase the yellow line depicting the signal during the “A” blocks, decrease the signal during the “B” block, and bring the signal back to a baseline during the unlabeled periods.

FIGS. 12C-D are the event related average and the time course of the signal change during the second training run. The subject improves after a single run as illustrated by both the event related average graph in FIG. 12C and the time course data in FIG. 12D.

### Example 5

### Real-Time fMRI Neurofeedback in the Amygdala

The amygdala is a bilateral, almond-shaped group of neurons located deep within the medial temporal lobes of the brain and is believed to perform a primary role in the processing and memory of emotional reactions. As such, the amygdala is another target of rt-fMRI applied to psychiatric illness.

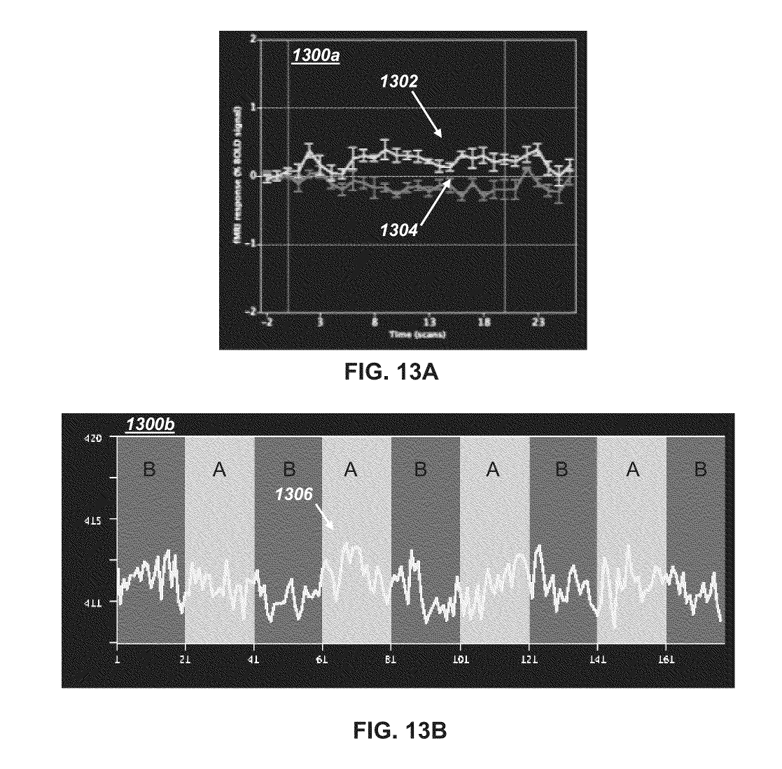

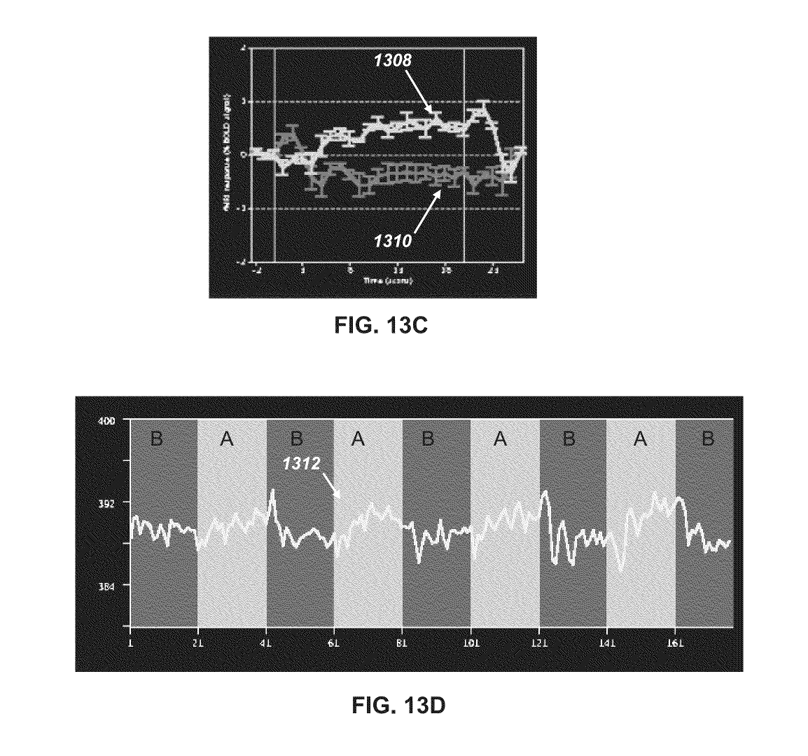

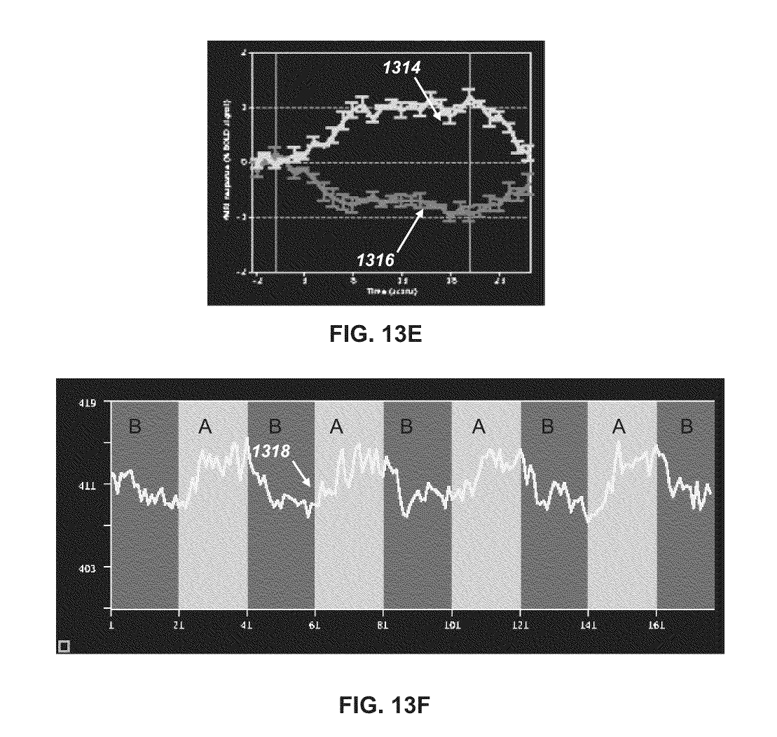

FIG. 13A depicts a graph 1300a of the event related average of the increase blocks (line 1302) compared to the decrease blocks (line 1304) and FIG. 13B depicts a graph 1300b of the time course 1306 of activation in the region of interest (the amygdala) during the first rt-fMRI acquisition. FIGS. 13BC-13D and FIGS. 13E-13F are the results from the second and third rt-fMRI neurofeedback training runs, respectively. As in previous examples, during the blocks labeled A, the subject was supposed to try and increase the activation, and during the blocks labeled B, she was supposed to decrease activation. The subject in this study was able to improve self-control over activation with practice and becomes much better at making the yellow line go up during A blocks and go down during B blocks.

FIGS. 13A-13B corresponds to the initial rt-fMRI acquisition, without supplying feedback to the subject. FIGS. 13C-13D depicts the signal activation in the selected region of interest during the first rt-fMRI neurofeedback run targeting the right amygdala, and FIGS. 13E-13F depicts the case for the second rt-fMRI neurofeedback training run targeting the right amygdala.

These data demonstrate how rt-fMRI neurofeedback can train a subject to control activation in the amygdala, one of the brain regions thought to be overactive in PTSD, anxiety disorders, addictions, and some mood disorders. Amygdala/hippocampal/prefrontal connections are thought to be mediated by NMDA receptors and modulated by d-cycloserine. This circuit helps control fear extinction and is thought to be the circuit modified in the Ressler paper using d-cycloserine to help improve fear extinction in acrophobia. The amygdala will be a prime target for our pilot studies using d-cycloserine to enhance rt-fMRI. That we can successfully target the amygdala with rt-fMRI demonstrates an important aspect of feasibility.

### Example 6

### Measuring Real-Time fMRI Neurofeedback after Administration of an Agent that Enhances NMDA Receptor-Mediated Neurotransmission

In this example, real-time fMRI neurofeedback is combined with the administration of D-cycloserine to enhance NMDA receptor-mediated neurotransmission.

The primary endpoint in the previously-described examples is achieving self-control over brain activation, with changes in brain activation signal at a single time point being the measure of success. The focus of the present example is improving the degree of signal enhancement, increasing the persistence of the self-modulatory control, and defining the functional consequence of obtaining the ability to self-regulate brain activation in a given area or circuit of the brain.

This protocol describes a blinded, group comparison, placebo controlled pilot trial of D-cycloserine administered in conjunction with rt-fMRI neurofeedback to demonstrate that addition of an NMDA receptor partial agonist can enhance rt-fMRI neurofeedback. Realtime fMRI neurofeedback can effectively train a subject to achieve an average 1% signal change in a selected region of interest (ROI). The addition of the NMDA partial agonist, d-cylcoserine, administered in single doses (e.g., 50 mg, 100 mg, 250 mg, 500 mg, or other dose) just prior to the training regimen enables a subject to increase more effectively the percent signal change under voluntary control. For example, a more effective increase is a change in BOLD signal larger than 1% beyond that achieved by the control population receiving only a placebo in conjunction with rt-fMRI neurofeedback.

Each subject has two separate training sessions scheduled 1 to 2 weeks apart, with a third follow-up evaluation, so that this study also shows that D-cycloserine can more effectively consolidate the learning achieved with rt-fMRI neurofeedback. Repeated measures analysis and group comparisons are conducted to determine D-cycloserine's effect on the persistence of the rt-fMRI neurofeedback training.

The study includes four groups:
1. A control group receiving two training sessions of rt-fMRI neurofeedback in conjunction with a placebo.
2. An active group receiving two training sessions of rt-fMRI neurofeedback in conjunction with 50 mg of D-cycloserine administered one hour prior to each scanning session.
3. An active group receiving two training sessions of rt-fMRI neurofeedback in conjunction with 250 mg of D-cycloserine one hour prior to each scanning session.
4. An active group receiving two training sessions of rt-fMRI neurofeedback in conjunction with 500 mg of D-cycloserine one hour prior to each scanning session.

The rt-fMRI neurofeedback sessions target regions of interest (ROI) within the amygdala, hippocampus, insula, and cortical regions that activate in response to an initial functional localizing scan.

D-cycloserine (trade name Seromycin®), manufactured by Eli Lilly Company of Indianapolis, Ind., is administered to the patients at a dosage between 50 mg and 500 mg as indicated.

Outcome Measures and Procedures of Study

The primary outcome measures include the following:
a) Percent signal change under voluntary control as measured by the change in BOLD activation in the ROI comparing the increase blocks to the decrease blocks.
b) Change in the number of voxels within the region of interest correlating with the increase/decrease task that reach statistical significance.
c) A measure of the training persistence by comparing the final training session to the initial training session run using the equation:

[(fMRI_{increase}−fMRI_{decrease})_{final}−(fMRI_{increase}−fMRI_{decrease})ᵢₙᵢₜᵢₐₗ]

Secondary outcome measures include the following:
a) Self report of activation strategies.
b) Change in self-reported scores on the visual analogue scales (VAS) of mood/anxiety/cravings comparing pre to post training session assessments.
c) Change in performance comparing pre to post training sessions on any of the cognitive tasks completed.

The study visit procedures include the following:
1) Subject consenting.
2) Completion of pre-MR screening form.
3) Completion of pregnancy screen for women of child-bearing potential and completion of a urine pregnancy test if pregnancy status is uncertain or at the discretion of the physician.
4) Randomization into one of the four study groups (50 mg, 250 mg, 500 mg DCS or placebo).
5) Oral administration of the study medication.
6) Completion of mood/anxiety rating scales and, if indicated, one of the cognitive tasks (described below).
7) Structural MRI scan.
8) Functional MRI scan to functionally localize and select a region of interest (ROI).
9) Real-time fMRI neurofeedback instructions.
10) Real-time fMRI neurofeedback training sessions (5-7 sessions, each lasting 5-7 minutes).
11) Repeat of the mood/anxiety rating scales and, if completed prior to scanning, repeat completion of the cognitive task.
12) Exit interview

During the fMRI localizing scan and during feedback, subjects are presented with one or more of a visual, auditory, or olfactory stimulus. Alternatively or in addition, subjects are asked to perform one or more of a mood induction, a rating scale, a simple motor task, a continuous performance task, a Stroop interference task, a facial emotion discrimination task, or a rapid visual information processing task.

The subjects are instructed to perform tasks that will engage the brain areas under investigation. If visual stimulation is conducted, different images will be either projected onto a video screen that can be seen in a mirror, or presented in a set of special goggles. During the presentation of the images, the subject is asked to think about the stimuli or cues presented, and to try to avoid thinking about other things, as this may complicate the interpretation of the scans. They are asked to respond to or rate the visual cues in an effort to activate the area of the brain being targeted by the neurofeedback training. The images are pictures of people with different facial expressions, images of food, smoking, or beverages, or of scenes from the international affective picture system (IAPS). Auditory cues may be music or musical notes, mechanical sounds, human or animal vocalizations, or selections from the International affective digital sound library. Olfactory cues are odorants characterized to illicit pleasant, unpleasant, or craving responses. Tactile cues range from barely discernable to painful, but not harmful, vibrations, pressure, temperature changes, or other tactile sensations. These cues are initially used to induce brain activation in the region to be targeted by neurofeedback. The cue-induced activation of a region refines and individualizes the anatomical region selection and assists the subject's self-modulating efforts by initiating activity in a given region of interest.

The stimulus presentation computer is located in the scanner control room. The video signal is sent to a projector that projects the pictures onto a screen located at the back of the scanner. The subject views the pictures on the screen by looking at a mirror that is mounted on the head coil, directly in front of the subject's eyes.

For studies of brain regions involved in emotional processing, subjects are asked to attempt to experience different types of moods while they are in the scanner. This involves remembering emotional events in their lives. Both positive and negative events may be remembered.

The brain's response in a given region or circuit to stimuli such as the cues described above or tasks such as mood induction is determined during the course of the scan, and subjects are given an indication of the strength of this response in the form of a feedback signal. The feedback signal is a representation of the neuronal activity in a given region or circuit of the brain. The feedback signal is transmitted to the subject via either visual, auditory, tactile, or olfactory modalities. This indication is given by a change in the level of a line graph, or the color, shape, speed or size of an object they can see on the video screen, by changing the tone, volume, or frequency of a sound that they hear over the headphones, by adjusting an odor administered via an MRI compatible olfactometer, or by varying the temperature, vibration, pressure, or some other characteristic of a tactile stimulus.

Subjects are asked to rate their mood, level of anxiety, sense of craving, and/or general sense of well being before and after rt-fMRI neurofeedback. They are also asked to perform some psychological tasks to get either baseline measurements or to assess the effects of biofeedback such as the continuous performance task, the Stroop interference task, the emotional facial discrimination task, and the rapid visual information processing task. They are asked to perform these tasks both before and after the scan to help monitor the effects of the rt-fMRI neurofeedback. These tasks last between 15 and 30 minutes.

Subjects are instructed to rest quietly in the scanner. They are under constant visual and audio observation by the scanner technologist. Total scan time per exam will not exceed 2 hours. Subjects are asked to return, usually 1 to 2 weeks after the first session, for a second visit. Subjects are also asked to return for a third follow-up visit.

Second visit procedures are the same as the first visit described except that subjects remain in the study group to which they were originally assigned.

Follow-up visit procedures include the following:
1. Subject consenting
2. Completion of pre-MR screening form
3. Completion of pregnancy screen for women of child-bearing potential and completion of a urine pregnancy test if pregnancy status is uncertain or at the discretion of the physician.
4. Structural MRI scan
5. Functional MRI scan to functionally localize and select region of interest (ROI)
6. Real-time fMRI neurofeedback instructions
7. Real-time fMRI neurofeedback training sessions (5-7 sessions, each lasting 5-7 minutes).
8. Exit interview

Details of Magnetic Resonance Measurements

MR acquisitions are performed on any human MR scanner (e.g., a scanner provided by Siemens Medical Solution, Bruker BioSpin, or GE Healthcare) of any clinical magnetic field strength (e.g., 1.5 T, 3.0 T, 4.7 T, 7 T, 8 T). Radiofrequency (RF) pulses and gradient waveforms should be within FDA safety limits for specific absorption rate (SAR) and the time-rate change of the magnetic fields (dB/dt) and should operate within FDA acoustic limits. RF body and surface coils are provided with the system. In addition, standard EPI (Echo Planar Imaging) and functional MRI with feedback options can be included.

Structural MRI uses MR imaging to provide anatomic information of the brain and body. These data provide morphometric and volumetric information.

Specific scan parameters for this study varies depending on the specific brain region under examination. Examples of anatomic MRI sequences include:
1. T1: 3D MPRAGE sequence
   TE/TR/TI=3.97 ms/2.1 s/1.1 s;
   12° flip angle;
   256×256 on 256 FOV;
   128 slices;
   1.5 mm thickness with 0 gap;
   1 average;
   sagittal scan;
   scan duration: about 9 minutes;
2. T2: double echo fast spin echo sequence
   TE/TR=28 ms/84 ms/7110 ms;
   echo-train length=5;
   240×87.5 matrix on 220×192 5 mm FOV;
   60 slices;
   3 mm thickness with 0 gap;
   1 average;
   axial scan;
   duration=5 minutes to 7 minutes;
3. FLAIR: fast spin echo sequence with inversion
   TE/TR/TI=90 ms/8 s/2.5 s;
   echo-train length=13;
   384×252 matrix on 220×192 5 mm FOV;
   21 slices;
   5 mm thickness with a 2 mm skip;
   1 average;
   axial scan;
   duration=about 5 minutes;

Functional MRI uses MR imaging to measure the quick, tiny metabolic changes that take place in an active part of the brain. fMRI looks closely at the anatomy of the brain, and can help determine precisely which part of the brain is handling critical functions such as thought, speech, movement, and sensation. This information helps provide a better understanding of the brain and body, their disorders and treatments. Examples of fMRI sequences are BOLD, Diffusion, and T2 Relaxometry. Specific scan parameters vary but are within FDA clinical safety limits. Real time functional MRI (rt-fMRI) is performed as discussed below.

BOLD scanning is the name given to the standard functional MRI acquisition that uses gradient echo, EPI sequences. It is a non-contrast technique, based on a T2* weighted gradient echo pulse sequence, which has demonstrated sensitivity to the local concentration of paramagnetic deoxyhemoglobin.

During acquisition, visual stimulation is provided to the subject, along with an assignment of cognitive, motor, or mood induction tasks to perform. Typically acquired using the head RF coil, BOLD scans can also use a 5″ GP surface coil or other special head RF coils. All scan planes can be used for these acquisitions. Current 3 T parameters are: TE=21 msec, TR=1.5 seconds, matrix either 64×64 or 128×128 on a 20 cm FOV, with a slice thickness ranging from 3 mm to 10 mm. Frequency direction is R/L (in sagittal planes it is S/I). Scan time is typically 2 minutes, commonly 30 seconds to 5 minutes, but longer scan times, e.g., 7 minutes, 10 minutes, 17 minutes) can be used.

In fMRI scans, the brain's response to stimuli or tasks in some brain region is determined during the course of the scan, and subjects are given an indication of strength of this response. This indication is given by a change in the color, shape or size of something they can see on the video screen, by changing the tone, volume, or frequency of a sound that they hear over the headphones, by adjusting an odor administered via an MRI compatible olfactometer, or by varying the temperature, vibration, pressure, or some other characteristic of a tactile stimulus. Subjects are asked to try to change the strength of the brain response by performing certain mental tasks and/or exploring different cognitive strategies. This includes, changing the task they are already performing, or thinking of some image (for example, imagining they are riding in a car, and having the car speed up or slow down to change the strength of the brain activity). This process is harmless, and subjects can stop at any time.

The Turbo-BrainVoyager™ software package is used to enable rt-fMRI studies. The software does not alter fMRI scan parameters from those approved for use on the 3 T system, and the study is completely noninvasive. Turbo-BrainVoyager™ is a data analysis package that accelerates image processing such that processed images showing brain response patterns to stimuli and feedback can be shown to subjects within seconds. This allows subjects to quickly review, react to, and actively modify their brain response patterns.

Sample Size and Power Calculation

Sample size calculations are based on detecting differences between treatment groups and placebo in BOLD activation signal, our primary outcome measure. In this example, each study group includes 12 subjects:

$$N = \frac{{( {Z}_{α} )}^{2} * 2 * {( σ )}^{2}}{{( d )}^{2}} = \frac{{( 1.96 )}^{2} * 2 * {( 0.30 )}^{2}}{{( 0.25 )}^{2}} = 11.0$$

in which Z_{α} represents the α-level critical value of the standard normal distribution, a represents the estimated standard deviation, and d represents the anticipated difference. We estimate that a sample of 48 subjects; a placebo group, a 50 mg DCS group, a 250 mg DCS group and a 500 mg DCS group all receiving standard rt-fMRI feedback in addition to the study medication. Four groups of 12 individuals allows a determination of whether outcome measure differences are statistically significant.

### Example 7

### Measuring Real-Time fMRI Neurofeedback after Administration of an Agent that Enhances NMDA Receptor-Mediated Neurotransmission for Anxiety Disorders

Phobic disorders are characterized by an excessive fear of a specific object or situation that is triggered by the presence or anticipation of encountering such a situation or object. The fear is extreme, can generate panic attacks, and inhibits normal functioning.

Treatment of phobic disorders generally relies on a combination of anti-anxiety medications and behavioral therapy. Real-time fMRI neurofeedback targeting the amygdala and the hippocampus and administration of DCS can enhance the acquisition of extinction in individuals with anxieties (e.g., post-traumatic stress disorders, panic disorders, phobias, generalized anxiety disorders, obsessive compulsive disorder).

Obsessive compulsive disorder (OCD) is a type of anxiety and can be a life altering illness characterized by intrusive obsessional thoughts and compulsive, often ritualistic behaviors. Typically, obsessional thoughts are triggered by environment cues and commonly take the form of either mental or physical contamination. These unwanted obsessions generate a state of extreme anxiety and hyperarousal. Individuals with OCD remain in a state of extreme anxiety and hyperarousal until their particular compulsive behavior or ritual somehow eradicates or cleanses them of their obsession.

The prevalence of OCD is thought to be between 1 and 3% of the population. Current treatment for OCD relies primarily on a combination of pharmacotherapy and cognitive behavioral interventions and is commonly inadequate. Cognitive behavioral therapy relies primarily on exposure to the anxiety producing agent, and then response prevention. The response prevention is an inhibition of the compulsive behavior and is key to the success of the therapy. Response prevention enables the individual to extinguish their anxiety produced by the triggering or offending cue through habituation. This study evaluates if the process of habituation can be augmented by rt-fMRI neurofeedback in combination with NMDA receptor activation.

This study involves four groups of subjects:
1. A control group receiving placebo and sham rt-fMRI neurofeedback
2. A control group receiving placebo and actual rt-fMRI neurofeedback
3. A control group receiving DCS and sham rt-fMRI neurofeedback
4. An active group receiving DCS and actual rt-fMRI neurofeedback

OCD symptoms are assessed prior to the MRI scanning session using a standard Yale Brown obsessive compulsive symptom checklist and visual analogue scales of anxiety, mood, and obsessionality.

Each of the four groups would receive DCS or placebo 1 hour prior to rt-fMRI neurofeedback or sham rt-fMRI training.

After anatomical scans are obtained, rt-fMRI neurofeedback training runs targeting the orbitofrontal/anterior cingulate/caudate/thalamic circuits are conducted over the course of a 1-2 hour scanning session.

Symptoms are reassessed after rt-fMRI training using checklist and visual analogue scales. Subjects return within 2 weeks to repeat the procedure. Subjects are followed up in one month to assess memory performance and rt-fMRI neurofeedback self-regulation ability without any medication.

Endpoints include:
1 Change of percent BOLD signal in the orbitofrontal/anterior cingulate/caudate/thalamic circuit between the increase blocks and decrease blocks in the first training session compared to the last training session.
2. Scores on the checklists and visual analogue scales.
3. Quality of life assessments.

### Specifics for Enhancement of Memory and Learning in Normal Controls

A specific aim of this study is to assess whether DCS in combination with rt-fMRI can enhance learning memory function by increasing an individual's ability to self regulate brain activation in the hippocampus and hippocampal/amygdalar circuits.

N-methyl-D-aspartate receptor (NMDA-R) mediated glutamate neurotransmission in the brain produces long term potentiation, a form of synaptic plasticity thought to be a key step in the cellular mechanism of learning and memory.

Study groups for this study can involve four groups of subjects:
1. A control group receiving placebo and sham rt-fMRI neurofeedback
2. A control group receiving placebo and actual rt-fMRI neurofeedback
3. A control group receiving DCS and sham rt-fMRI neurofeedback
4. An active group receiving DCS and actual rt-fMRI neurofeedback

Memory would be assessed prior to the MRI scanning session using a standard memory performance task. The three groups would receive DCS or placebo 1 hour prior to rt-fMRI neurofeedback or sham rt-fMRI training.

After anatomical scans are obtained, rt-fMRI neurofeedback training runs targeting the hippocampus and/or hippocampal circuits would be conducted over the course of a 1-2 hour scanning session.

Using a different version of a standard memory performance task, memory would be reassessed after the one hour training session is complete.

Subjects would return within 2 weeks to repeat the procedure. Subjects would be followed up in 1 month to assess memory performance and rt-fMRI neurofeedback self-regulation ability without any medication.

Endpoints for this study include:
1. Change of percent BOLD signal in the hippocampus between the increase blocks and decrease blocks in the first training session compared to the last training session.
2. Performance scores on the memory assessment tasks.

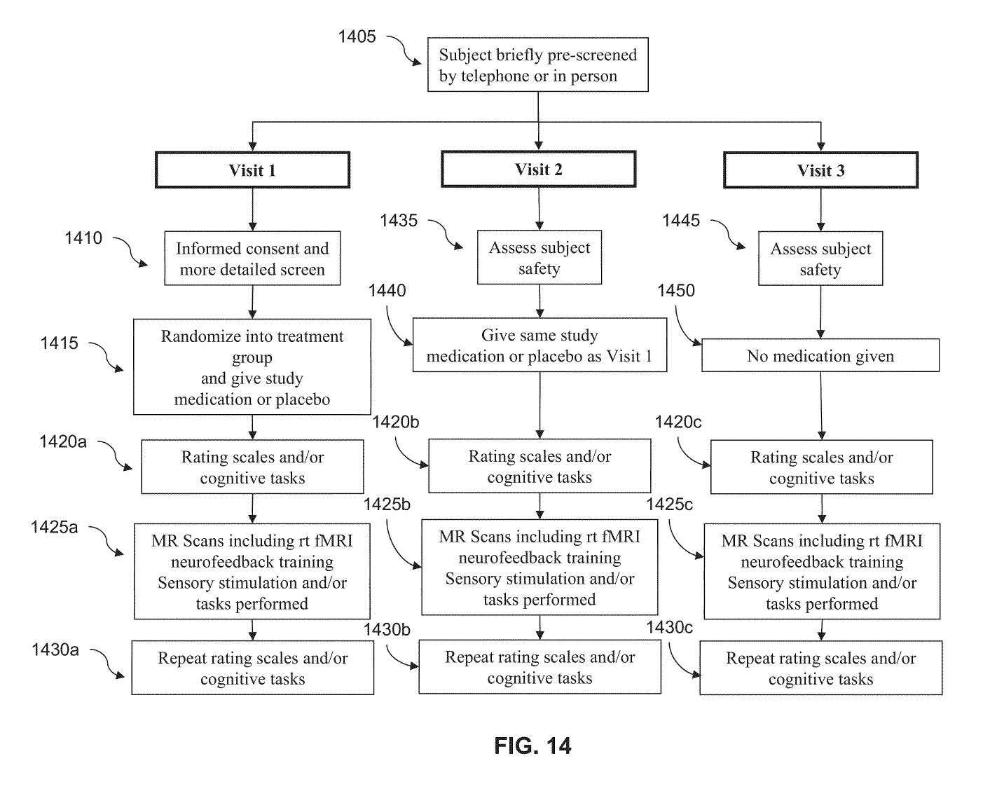

Referring now to FIG. 14, a flow chart summarizes example steps in studies conducted on patients. The patient or subject is briefly pre-screened (step 1405) in preparation of the study. The patient then visits a clinic or research facility a plurality of times. The example depicted in FIG. 14 shows the patient or subject making three separate visits.

During the first visit, the subject is provided with detailed information and his formal consent is taken (step 1410). If the subject is chosen to participate in the study, the subject is randomly put in a treatment group and given either the study medication at a certain dosage or placebo (step 1415). The subject is then given one or more cognitive tasks and/or a mood/anxiety rating scale is associated with the subject (step 1420a). Magnetic resonance (MR) scans including multiple runs of rt fMRI neurofeedback is performed on the subject (step 1425a). The fMRI localizes and selects a region or circuit of interest in the subjects' brain. The scans are performed in the presence of sensory stimulation and/or during the performance of the cognitive tasks. After the rt-fMRI neurofeedback training runs are completed, the mood/anxiety rating scales are repeated for the subject and/or the subject is asked to complete the cognitive tasks (step 1430a) assigned to them.

In a second visit, the safety of the subject is assessed (step 1435). If no safety threats are detected, the subject is administered the same medication or placebo administered during the first visit (step 1440). The subject then undergoes substantially the same procedures as described with respect to the first visit. These are denoted as steps 1420b, 1425b and 1430b and are substantially same as the steps 1420a, 1425a and 1430a, respectively.

In the third visit, the safety of the subject is assessed again (step 1445). Again, if no safety threats are detected, procedures substantially same as the earlier visits are repeated, except without the administration of the medication or placebo. In FIG. 14 these steps are denoted as 1420c, 1425c and 1430c are substantially same as the steps 1420a, 1425a and 1430a, respectively.

### Other Embodiments

It is to be understood that while the invention has been described, the foregoing description is intended to illustrate and not to limit the scope of the invention, which is defined by the scope of the appended claims. Other aspects, advantages, and modifications are within the scope of the following claims.

1. A method of training a subject to modify the subject's neuronal activity, the method comprising:
administering to the subject an effective amount of an agent that enhances NMDA receptor-mediated neurotransmission;
measuring levels of the subject's neuronal activity within a selected brain region over a predetermined period of time; and
providing feedback to the subject in the form of a signal derived via imaging the selected brain region to enable the subject to modify the subject's neuronal activity within the selected brain region.

2. The method of claim 1, wherein the agent comprises at least one of d-cycloserine, d-alanine, d-serine, glycine, or sarcosine.

3. The method of claim 1, wherein a first level of the subject's neuronal activity within the selected brain region is measured using a brain imaging technology.

4. The method of claim 3, wherein the brain imaging technology comprises at least one of functional magnetic resonance imaging, electroencephalography, magnetoencephalography, positron emission tomography, and single photon emission computed tomography.

5. The method of claim 4, wherein data from the brain imaging are analyzed in real time.

6. The method of claim 5, wherein the brain imaging comprises functional magnetic resonance imaging.

7. The method of claim 1, wherein the providing feedback to the subject comprises:
presenting to the subject a representation of a level of the subject's neuronal activity within the selected brain region at a first time;
providing the subject with a set of instructions configured to modify the subject's neuronal activity within the selected brain region; and
presenting to the subject a representation of a level of the subject's neuronal activity within the selected brain region at a second time.

8. The method of claim 7, wherein the representation comprises a visual, olfactory, auditory, or tactile stimulus.

9. The method of claim 7, wherein an interval between the first and second times is about 100 ms to 10 seconds.

10. The method of claim 1, further comprising administering one or more of a direct or indirect dopamine agonist, a cholinergic agent, a norepinephrine agent, a neuroendocrine agent a glial cell supporter, a glutamate/glutamine cycling facilitator, a nonspecific cognitive enhancer, a mitochondrial enhancing agent, and a histone deacetylase agent, alone or in combination with a NMDA receptor partial agonist.

11. The method of claim 1, wherein the feedback is provided in the form of a visual signal.

12. The method of claim 1, wherein the feedback is provided in the form of a tactile signal.

13. The method of claim 1, wherein the feedback is provided in the form of an olfactory signal.

14. The method of claim 1, wherein the feedback is provided in the form of an auditory signal.

15. A computer-readable medium storing a computer program for training a subject to modify the subject's neuronal activity, the computer program comprising instructions for causing a computer system to:
receive data indicative of levels of the subject's neuronal activity within a selected brain region over a predetermined length of time, wherein the subject is administered an effective amount of an agent that enhances NMDA receptor-mediated neurotransmission; and
provide feedback to the subject in the form of a signal derived via imaging the selected brain region to modify the subject's neuronal activity within the selected brain region based at least on the received data.

16. The medium of claim 15, wherein the computer program further comprises instructions for causing a computer system to:
present to the subject a representation of a level of the subject's' neuronal activity within the selected brain region at a first time;
provide the subject with a set of instructions configured to help modify the subject's neuronal activity within the selected brain region; and
present to the subject a representation of a level of the subject's neuronal activity within the selected brain region at a second time.

17. A system of training a subject to modify the subject's neuronal activity, comprising:
a scanner configured to acquire data indicative of levels of the subject's neuronal activity within a selected brain region over a predetermined period of time;
a processor configured to instruct a user to administer an effective amount of an agent that enhances NMDA receptor-mediated neurotransmission to the subject at the beginning of the predetermined period of time and to analyze the data acquired by the scanner; and
an output device configured to provide the subject guidance to modify the subject's neuronal activity within the selected brain region.

18. The system of claim 17, wherein the scanner is a magnetic resonance (MR) scanner.

19. The system of claim 17, wherein the scanner is configured to execute at least one of functional magnetic resonance imaging, electroencephalography, magnetoencephalography, positron emission tomography, and single photon emission computed tomography.

20. The system of claim 17, wherein the output device is configured to present the guidance by:
presenting to the subject a representation of a level of the subject's neuronal activity within the selected brain region at a first time;
providing the subject with a set of instructions configured to modify the subject's neuronal activity within the selected brain region; and
presenting to the subject a representation of a level of the subject's neuronal activity within the selected brain region at a second time.
